(19) United States
(12) Patent Application Publication
(10) Pub. No.: US 2021/0290085 A1
(43) Pub. Date: Sep. 23, 2021

(54) OPTICAL MEDICAL DETECTION DEVICE

(71) Applicant: PixArt Imaging Inc., Hsin-Chu City (TW)

(72) Inventors: Shih-Jen Lu, Hsin-Chu City (TW); Yang-Ming Chou, Hsin-Chu City (TW); Chien-Yi Kao, Hsin-Chu City (TW); Hsin-Yi Lin, Hsin-Chu City (TW); Chih-Ming Sun, Hsin-Chu City (TW); Chung-Yuo Wu, Hsin-Chu City (TW)

(21) Appl. No.: 16/822,024

(22) Filed: Mar. 18, 2020

(51) Int. Cl. A61B 5/0295; A61B 5/026; A61B 5/021; A61B 5/00; A61B 5/0205

(52) U.S. Cl. CPC A61B 5/0295; A61B 5/0261; A61B 5/02141; A61B 5/746; A61B 5/6824; A61B 5/7405; A61B 5/742; A61B 5/0022; A61B 5/02055; A61B 5/6829

(57) ABSTRACT

An optical medical detection device includes a first physiological sensor and a processor. The first physiological sensor is adapted to continuously output a first photoplethysmography (PPG) signal generated by a first skin area of an object, and further to optionally and continuously output a second photoplethysmography (PPG) signal generated by a second skin area of the object. The second skin area is different from the first skin area. The processor is electrically connected to the first physiological sensor. The processor is adapted to convert the first PPG signal and the second PPG signal into a physiological feature, and the physiological feature is at least one of a blood pressure ratio and a blood flow velocity.

### BACKGROUND OF THE INVENTION

### 1. Field of the Invention

The present invention relates to an optical medical detection device, and more particularly, to an optical medical detection device capable of continuously detecting the PPG signal to increase reliability and accuracy of cardiovascular evaluation.

### 2. Description of the Prior Art

A conventional barometric sphygmomanometer is extensively applied in the hospital or the health examination center. The conventional barometric sphygmomanometer is large-sized equipment put on the table, and the user ties the tourniquet of the conventional barometric sphygmomanometer on the arm or the leg, and then the tourniquet is filled by gaseous matter to measure blood pressure of the user. If the user intends to measure the blood pressure of another limb, a waiting period of exhausting the gaseous matter in the tourniquet is necessary, and a current physical sign of the user may be different from a previous physical sign due to time difference, so that an ankle-brachial index and a pulse wave velocity measured by the conventional barometric sphygmomanometer has drawbacks of inconvenient operation and poor accuracy.

### SUMMARY OF THE INVENTION

The present invention provides an optical medical detection device capable of continuously detecting the PPG signal to increase reliability and accuracy of cardiovascular evaluation for solving above drawbacks.

According to the claimed invention, an optical medical detection device includes a first physiological sensor and a processor. The first physiological sensor is adapted to continuously output a first photoplethysmography (PPG) signal generated by a first skin area of an object, and further to optionally and continuously output a second photoplethysmography (PPG) signal generated by a second skin area of the object. The second skin area is different from the first skin area. The processor is electrically connected to the first physiological sensor. The processor is adapted to convert the first PPG signal and the second PPG signal into a physiological feature, and the physiological feature is at least one of a blood pressure ratio and a blood flow velocity.

According to the claimed invention, the optical medical detection device further includes a second physiological sensor electrically connected to the processor and adapted to continuously output the second PPG signal generated by the second skin area. The first physiological sensor and the second physiological sensor synchronously output the first PPG signal and the second PPG signal at each point of time during a working period of the optical medical detection device. At least one of the first physiological sensor and the second physiological sensor is an array PPG sensor.

According to the claimed invention, the optical medical detection device further includes at least one temperature sensor electrically connected to the processor and adapted to acquire a temperature value around the first skin area or the second skin area. The processor outputs an alarm notice by referring to the temperature value and the physiological feature. The at least one temperature sensor comprises two temperature sensors electrically connected to the processor and adapted to acquire temperature values respectively around the first skin area and the second skin area, the processor outputs an alarm notice by referring to the two temperature values and the physiological feature.

According to the claimed invention, the optical medical detection device further includes a memory electrically connected to the processor and adapted to storage a plurality of first PPG signals and second PPG signals acquired at all points of time during a working period of the optical medical detection device.

According to the claimed invention, the first PPG signal and the second PPG are respectively converted into two blood pressure values from respective limbs of the object, and the blood pressure ratio is an ankle brachial index (ABI) result via a comparison between the two blood pressure values. Or, the first PPG signal and the second PPG are respectively converted into two reaching time from respective limbs of the object, and the blood flow velocity is a pulse wave velocity (PWV) result via a comparison between a total blood vessel length of the object and difference in foresaid reaching time.

According to the claimed invention, the processor outputs an alarm notice in response to the physiological feature conforming to a specific condition. The specific condition represents the physiological feature is out of a predefined range; or, the specific condition represents difference between an initial feature and the physiological feature is greater than a predefined threshold, and the initial feature and the physiological feature are acquired in respective working periods of the optical medical detection device.

According to the claimed invention, the alarm notice is sent to a recorded mailbox or a recorded medical treatment system. Further, the alarm notice is an acoustic signal or a visual signal generate by the optical medical detection device. The alarm notice is a detection result of the optical medical detection device, or a proposal of dealing with the detection result.

The optical medical detection device of the present invention designed as the portable apparatus can be convenient to execute the cardiovascular evaluation anytime and anywhere, and the continuous and synchronous detection of the ABI result and the PWV result can efficaciously increase the reliability and the accuracy of the cardiovascular evaluation via the optical detection technology. The present invention can monitor the blood microcirculation, the vascular obstruction and the vascular sclerosis at the same time by the optical medical detection device, and have advantages of the high accuracy, the preferred ease to use, and the inexpensive equipment cost.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a functional block diagram of an optical medical detection device according to a first embodiment of the present invention.

FIG. 2 is a diagram of the optical medical detection device wore by a user according to the first embodiment of the present invention.

FIG. 3 is a functional block diagram of an optical medical detection device according to a second embodiment of the present invention.

FIG. 4 is a diagram of the optical medical detection device wore by the user according to the second embodiment of the present invention.

### DETAILED DESCRIPTION

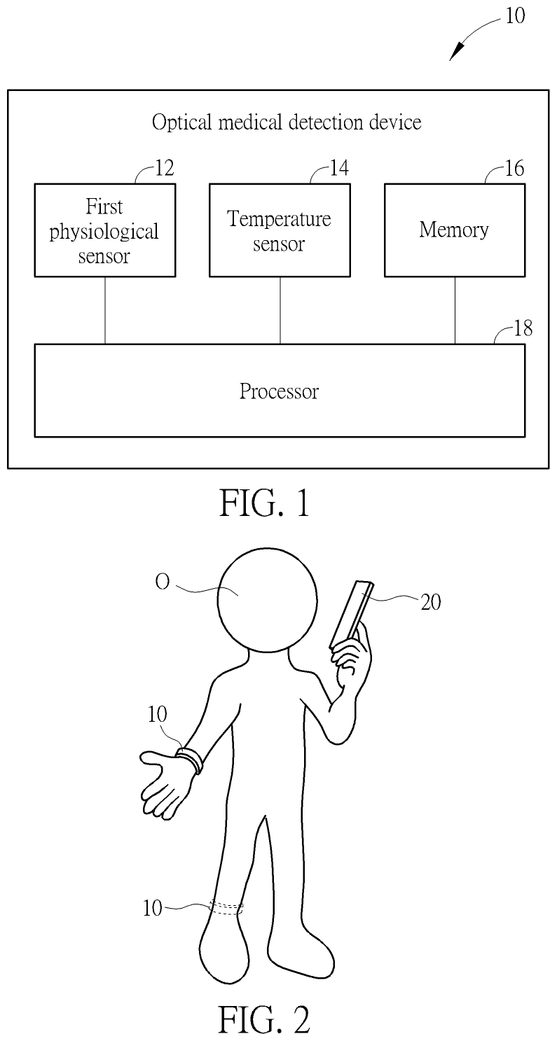

Please refer to FIG. 1 and FIG. 2. FIG. 1 is a functional block diagram of an optical medical detection device 10 according to a first embodiment of the present invention. FIG. 2 is a diagram of the optical medical detection device 10 wore by a user according to the first embodiment of the present invention. The optical medical detection device 10 can include one physiological sensor (such as a first physiological sensor 12), a temperature sensor 14, a memory 16 and a processor 18 electrically connected to each other. The first physiological sensor 12 is put on a skin area of an object O, and used to continuously output a photoplethysmography (PPG) signal generated by the skin area. The object O can be the user who wears the optical medical detection device 10, and the optical medical detection device 10 can be a portable apparatus linked with a smart communication apparatus 20 in a wireless manner.

The temperature sensor 14 can be an optional unit used to acquire a temperature value around the skin area where the first physiological sensor 12 is put. The memory 16 can be used to storage information about the PPG signal from the first physiological sensor 12 and the temperature value from the temperature sensor 14 continuously sensed at all points of time during a working period of the optical medical detection device 10. In the first embodiment, the first physiological sensor 12 preferably can be an array PPG sensor. The first physiological sensor 12 can be alternately put on two limbs to output a first PPG signal generated by a first skin area of an upper limb (such as an arm) and a second signal generated by a second skin area of a lower limb (such as a foot). The processor 18 can convert the first PPG signal and second PPG signal into a physiological feature of the object O, such as a blood pressure ratio and/or a blood flow velocity.

For example, the first PPG signal and the second PPG can be respectively converted into two blood pressure values from the upper limb and the lower limb of the object O, and the blood pressure ratio can be an ankle-brachial index (ABI) result via a comparison between the two blood pressure values. Further, the first PPG signal and the second PPG can be respectively converted into two reaching time from the upper limb and the lower limb of the object O, and the blood flow velocity can be a pulse wave velocity (PWV) result via a comparison between a total blood vessel length of the object O and difference in the foresaid reaching time. If the physiological feature conforms to a specific condition, the processor 18 can outputs an alarm notice for reminder.

The specific condition can represent the physiological feature is out of a predefined range. For instance, a blood vessel of the user is healthy in response to the ABI result around 0.901.30, and the blood vessel may be slightly blocked in response to the ABI result around 0.500.90, and the blood vessel may be severely blocked in response to the ABI result around 0.000.50, so that a healthy pressure of the blood vessel (which may interpret degree of vascular obstruction) can be ranged from 0.90 to 1.30; further, a risk of coronary artery disease is high in response to the PWV result is greater than 1400 cm/sec, and a healthy velocity of blood transmission (which may interpret degree of vascular sclerosis) can be ranged from 1350 cm/sec to 1450 cm/sec. The temperature value from the temperature sensor 14 can be auxiliary data for defining the alarm notice. Generally, the temperature sensor 14 can detect the temperature value of the lower limb; if the ABI result is decreased, a section of the lower limb between the first physiological sensor 12 and a toe has the decreased temperature value, so as to improve reliability and accuracy of cardiovascular evaluation accordingly.

In addition, the specific condition can represent difference between an initial feature and the physiological feature is greater than a predefined threshold. The predefined threshold can be pre-stored in the memory 16 and designed in accordance with an actual demand The first physiological sensor 12 can sense the initial feature (which means an initial PPG signal) at a particular moment of time the optical medical detection device 10 is just wore by the object O. The sensed initial feature can be reference stored in the memory 16. Since then, the first physiological sensor 12 can continuously output the first and second PPG signals; the optical medical detection device 10 can compare the initial feature with each of the PPG signals, and decide the physiological feature conforms to the specific condition when the said difference is greater than the predefined threshold. Thus, the initial feature is acquired in an early part of an initial actuation period, and the physiological feature is acquired in the working period of the optical medical detection device 10.

The alarm notice can be a variety of warning. For instance, the alarm notice can be an acoustic signal generated by a speaker (which is not shown in the figures) of the optical medical detection device 10, or a visual signal generate by a display (which is not shown in the figures) of the optical medical detection device 10. Content of the alarm notice may be a detection result of the optical medical detection device 10, for reminding whether the degree of the vascular obstruction and the vascular sclerosis are over a tolerance; the content of the alarm notice further may be a proposal of dealing with the detection result, such as first aid of cardiovascular diseases resulted from the severe vascular sclerosis and the severe vascular obstruction. Moreover, the alarm notice can be a statistic message sent to a recorded mailbox or a recorded medical treatment system. The alarm notice sent to the recorded mailbox may be reported as medical test; the alarm notice sent to the recorded medical treatment system may mean calling an ambulance.

The physiological feature may be extremely changed in sudden if the object O is suffered from scare or pain, and a conventional barometric sphygmomanometer which detects the physiological feature at one point of time may not accurately acquire an actual physiological feature of the user. In the first embodiment, the optical medical detection device 10 can be put on the upper limb and then the lower limb of the object O for detecting continuous data of the ankle-brachial index and the pulse wave velocity and the temperature value within the working period via optical detection technology. The optical medical detection device 10 can monitor variation of sensor data during short-term or long-term duration within the working period, so as to compute a mean value of all the sensor data to be the physiological feature, or to compute a mean value of the sensor data excluding extreme data to be the physiological feature.

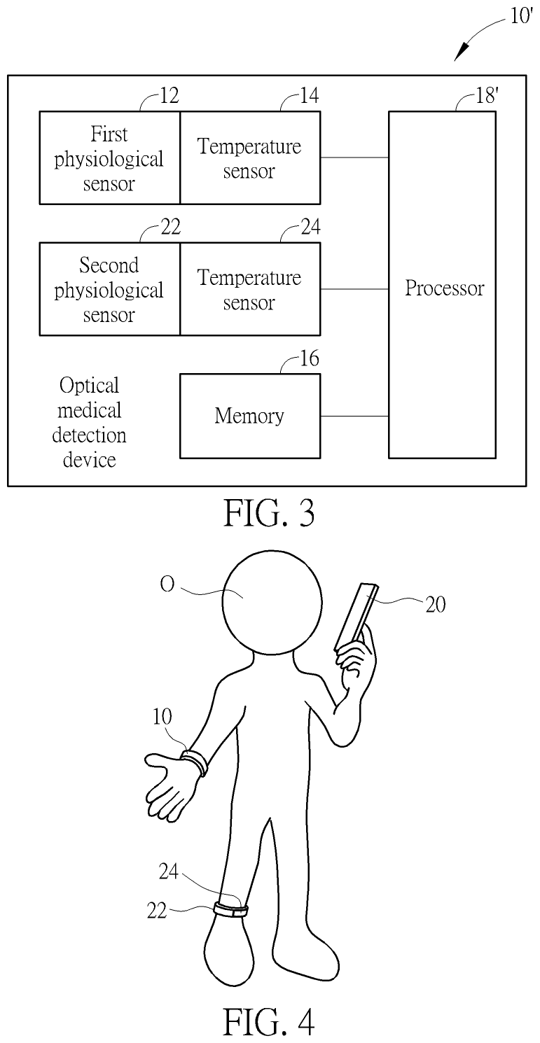

Please refer to FIG. 3 and FIG. 4. FIG. 3 is a functional block diagram of an optical medical detection device 10′ according to a second embodiment of the present invention. FIG. 4 is a diagram of the optical medical detection device 10′ wore by the user according to the second embodiment of the present invention. In the second embodiment, elements having the same numeral have the same function, and a detailed description is omitted herein for simplicity. The optical medical detection device 10′ can include two physiological sensors (such as the first physiological sensor 12 and a second physiological sensor 22), two temperature sensors 14 and 24, the memory 16 and the processor 18′ electrically connected to each other. The second physiological sensor 22 and the related temperature sensors 24 may be electrically connected to the processor 18′ in a wire manner or a wireless manner.

The first physiological sensor 12 can continuously output the first PPG signal generated by the first skin area (on the upper limb) of the object O, and the temperature sensor 14 can acquire the temperature value around the first skin area. The second physiological sensor 22 can continuously output the second PPG signal generated by the second skin area (on the lower limb) of the object O, and the temperature sensor 24 can acquire the temperature value around the second skin area. It should be mentioned that at least one of the first physiological sensor 12 and the second physiological sensor 22 can be the array PPG sensor.

In the second embodiment, the first physiological sensor 12 and the second physiological sensor 22 are respectively tied on the upper limb and the lower limb, so that the first PPG signal output by the first physiological sensor 12 and the second PPG signal output by the second physiological sensor 22 can be synchronous at each point of time during the working period of the optical medical detection device 10′, and accuracy of the physiological feature can be effectively increased. Difference between the foresaid two embodiments is: the first embodiment includes an assembly of one physiological sensor and one temperature sensor, which can be put on the one limb and then another to respectively acquire the first PPG signal and the second PPG signal, and has advantages of reduced cost, easy operation and accurate evaluation; the second embodiment includes two assembly individually with the physiological sensor and the temperature sensor, which can detect the first and second PPG signals from the upper limb and the lower limb at the same time, so that the reliability and the accuracy of the cardiovascular evaluation can be increased accordingly.

The optical medical detection device of the present invention can be applied to demands of regular measurement and instant measurement. In the regular measurement, the user may utilize the optical medical detection device to detect the blood pressure (such as detecting blood microcirculation) once or twice a day. If the detection result is abnormal, functions of detecting the ankle-brachial index and the pulse wave velocity can be actuated manually by the user or automatically by the optical medical detection device, and the optical medical detection device can transmit diagnostic reference to the recorded mailbox and/or the recorded medical treatment system. Besides, the detecting functions of the ankle-brachial index and the pulse wave velocity can be actuated twice or several times a year, and the abnormal detection result can be transmitted to the recorded mailbox and/or the recorded medical treatment system as the diagnostic reference. As the user has unilateral limb pain, the instant measurement can actuate functions of detecting limb temperature, the ankle-brachial index and the pulse wave velocity, and the detection result may drive calling of the ambulance.

Comparing to the prior art, the optical medical detection device of the present invention designed as the portable apparatus can be convenient to execute the cardiovascular evaluation anytime and anywhere, and continuous and synchronous detection of the ABI result and the PWV result can efficaciously increase the reliability and the accuracy of the cardiovascular evaluation via the optical detection technology. The present invention can monitor the blood microcirculation, the vascular obstruction and the vascular sclerosis at the same time by the same optical medical detection device, and have advantages of high accuracy, preferred ease to use, and inexpensive equipment cost.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An optical medical detection device, comprising:
a first physiological sensor adapted to continuously output a first photoplethysmography (PPG) signal generated by a first skin area of an object, and further to optionally and continuously output a second photoplethysmography (PPG) signal generated by a second skin area of the object, the second skin area being different from the first skin area; and
a processor electrically connected to the first physiological sensor, the processor being adapted to convert the first PPG signal and the second PPG signal into a physiological feature, the physiological feature being at least one of a blood pressure ratio and a blood flow velocity.

2. The optical medical detection device of claim 1, further comprising:
a second physiological sensor electrically connected to the processor and adapted to continuously output the second PPG signal generated by the second skin area;
wherein the first physiological sensor and the second physiological sensor synchronously output the first PPG signal and the second PPG signal at each point of time during a working period of the optical medical detection device.

3. The optical medical detection device of claim 2, wherein at least one of the first physiological sensor and the second physiological sensor is an array PPG sensor.

4. The optical medical detection device of claim 1, further comprising:
at least one temperature sensor electrically connected to the processor and adapted to acquire a temperature value around the first skin area or the second skin area, the processor outputting an alarm notice by referring to the temperature value and the physiological feature.

5. The optical medical detection device of claim 4, wherein the at least one temperature sensor comprises two temperature sensors electrically connected to the processor and adapted to acquire temperature values respectively around the first skin area and the second skin area, the processor outputs an alarm notice by referring to the two temperature values and the physiological feature.

6. The optical medical detection device of claim 1, further comprising:
a memory electrically connected to the processor and adapted to storage a plurality of first PPG signals and second PPG signals acquired at all points of time during a working period of the optical medical detection device.

7. The optical medical detection device of claim 1, wherein the first PPG signal and the second PPG are respectively converted into two blood pressure values from respective limbs of the object, and the blood pressure ratio is an ankle brachial index (ABI) result via a comparison between the two blood pressure values.

8. The optical medical detection device of claim 1, wherein the first PPG signal and the second PPG are respectively converted into two reaching time from respective limbs of the object, and the blood flow velocity is a pulse wave velocity (PWV) result via a comparison between a total blood vessel length of the object and difference in foresaid reaching time.

9. The optical medical detection device of claim 1, wherein the processor outputs an alarm notice in response to the physiological feature conforming to a specific condition.

10. The optical medical detection device of claim 9, wherein the specific condition represents the physiological feature is out of a predefined range.

11. The optical medical detection device of claim 9, wherein the specific condition represents difference between an initial feature and the physiological feature is greater than a predefined threshold, and the initial feature and the physiological feature are acquired in respective working periods of the optical medical detection device.

12. The optical medical detection device of claim 9, wherein the alarm notice is sent to a recorded mailbox or a recorded medical treatment system.

13. The optical medical detection device of claim 9, wherein the alarm notice is an acoustic signal or a visual signal generate by the optical medical detection device.

14. The optical medical detection device of claim 13, wherein the alarm notice is a detection result of the optical medical detection device, or a proposal of dealing with the detection result.

15. The optical medical detection device of claim 1, wherein the optical medical detection device is a portable apparatus linked with a smart communication apparatus in a wireless manner.
